(19) United States
(12) Patent Application Publication
(10) Pub. No.: US 2005/0095700 A1
(43) Pub. Date: May 5, 2005

(54) Method for maintaining low shear in a bioprocessing system

(76) Inventors: Thomas Budzowski, Pottstown, PA (US); Curtis Graham, Philadelphia, PA (US); Shang-Chih Jan, Audubon, PA (US); Richard Siegel, Chester Springs, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/980,792

(22) Filed: Nov. 3, 2004

Related U.S. Application Data
(60) Provisional application No. 60/516,917, filed on Nov. 3, 2003.

(51) Int. Cl. C12N 5/02

(52) U.S. Cl. 435/325

(57) ABSTRACT

Methods for maintaining a low shear environment in a bioprocessing system are disclosed. The methods of the invention are useful for extending the time for which a bioprocessing system can be operated thereby maximizing production time and the amount of product that can be recovered from the system.

### CROSS-REFERENCE OF RELATED APPLICATION

This application claims the benefit of U.S. Provisional Application No. 60/516,917, filed Nov. 3, 2003.

### FIELD OF THE INVENTION This invention relates to the maintenance of a low shear environment in a continuous perfusion bioprocessing system.

### BACKGROUND OF THE INVENTION

Modern biological drugs are produced by bioengineered fully viable cells that use soluble nutrients as growth and energy sources to produce and secrete the desired end product in final form. Both prokaryotic and eukaryotic systems are known.

Large-scale culture of single cell bacteria, yeast and molds is highly developed and these cells can be grown in large volumes of vigorously agitated liquid medium without any significant damage due to their tough cell walls. Conversely, eukaryotic cells generally have cell membranes that cannot withstand excessive turbulent action without damage to the cells and must be continuously provided with a complex nutrient medium to support growth.

In continuous perfusion bioreactors for growing eukaryotic cells, the external medium becomes the source material for harvest of the end product as well as the nutrient source for continued cell growth. To effect the removal of soluble product from the cell suspension, the nutrient medium containing the soluble product must be continually removed from the cells. However, bioreactor vessels and cell separation components with internal moving parts may damage eukaryotic cells and also subject the cells to high fluid shearing stresses. Cell damage and shear stress results in cell death and cell growth inhibition leading to decreased cell density and product yields.

Some fluid shearing stresses can be quantified and are measured as shear rate with units of s⁻¹. Shear rate is related to shear flow stress and viscosity where shear rate (γ)=shear flow stress (t)/viscosity (μ). Shear flow stress can be generated by moving liquid past static cells, moving cells through static liquid or by moving the liquid and the cells simultaneously and is generally quantified in dynes/cm². Viscosity is measured in poise where 1 poise=1 dyne sec cm⁻²=100 centipoise (cp). The viscosity of water, one of the least viscous fluids known, is 0.01 cp. The viscosity of a typical suspension of eukaryotic cells in media is between 1.0 and 1.1 cp at a temperature of 25° C. Changes in density or temperature of a fluid can also contribute to its viscosity.

Other fluid shearing stresses are those resulting from turbulent flow in a tube such as flexible tubing, conduit or pipe. In developed laminar flow of a Newtonian fluid through a straight tube of diameter (d), the shear rate at the wall depends on the mean flow velocity. There is a tendency for the liquid to resist movement and fluid closest to a solid surface will resist movement to a greater extent thereby creating a boundary layer and a velocity gradient relative to the distance from the solid surface. The steepness of the velocity gradient is a function of the speed at which the liquid is moving and its viscosity. At some point, as the liquid flow rate through or around a container accelerates, the laminar flow rate overcomes the viscosity of the liquid and a smooth velocity gradient breaks down producing turbulent flow. Thomas et al. in Cytotechnology 15: 329-335, (1994) showed that cell lysis was more closely related to overall shear stress under turbulent conditions than to shear stress alone.

Integral to continuous perfusion systems is a cell retention device (CRD) providing a means for separating viable cells from the culture medium and returning the cells with fresh medium to the reaction vessel. CRDs include mechanical devices such as filters or membranes and non-mechanical devices such as gravity settlers, centrifuges, acoustic filters and dielectrophoresis apparatus.

A particularly effective method for separating cells and harvesting product is centrifugal separation of cells from medium with a spin filter device. Internal spin filters have been used as a low shear system for large-scale perfusion culture bioreactor based bioprocessing systems. Internal spin filter perfusion bioreactor cell culture apparatus are described in, e.g., U.S. Pat. Nos. 5,126,269 and 5,637,496. However, clogging of internal spin filters during the operation of a perfusion bioreactor limits the number of days that a perfusion cell culture based bioprocessing system can be operated.

An external spin filter (ESF) can also be used for harvesting product from a perfusion cell culture based bioprocessing system. ESF technology enables the change out of the ESF filter material during perfusion culture, thus extending the number of days a perfusion cell culture based bioprocessing system can be operated. Typically, the use of ESF for scaled-up production of proteins from a perfusion cell culture based bioprocessing system has been accomplished using a lobe pump for recirculation. However, the ESF creates significant shear stresses on those cells carried in the medium that pass through the pump and filter unit.

These major sources of shear stress can all negatively affect protein production in a perfusion cell culture based bioprocessing system. Thus, a need exists for methods that can maintain cell density in a eukaryotic cell culture bioprocessing system by controlling the major sources of shear forces in such systems.

### BRIEF DESCRIPTION OF THE DRAWINGS

FIG. 1 shows a bioprocessing system schematic.

FIG. 2 shows details of an external spin filter device.

FIG. 3 shows the effect of shear produced by a lobe pump on cell viability and density in a bioprocessing system.

FIG. 4 shows improved cell growth and viability produced by the use of a peristalitic pump in a low-shear bioprocessing system.

### SUMMARY OF THE INVENTION

The present invention provides a method for maintaining a low shear environment in a eukaryotic cell bioprocessing system comprising the steps of culturing a cell suspension in a vessel; removing a portion of the suspension from the vessel by the action of a peristaltic pump; delivering the portion of the suspension to an external cell retention device that separates the suspension into a permeate stream and a retentate stream wherein the shear rate in the external cell retention device is less than 3000 sec⁻¹; and returning the retentate stream to the vessel.

### DETAILED DESCRIPTION OF THE INVENTION

All publications, including but not limited to patents and patent applications, cited in this specification are herein incorporated by reference as though fully set forth.

The term “antibody” as used herein and in the claims is meant in a broad sense and includes immunoglobulin or antibody molecules including polyclonal antibodies, monoclonal antibodies including murine, human, humanized and chimeric monoclonal antibodies and antibody fragments.

The term “antibody-derived binding protein” means a molecule comprising a portion of an antibody that is capable of binding a second molecule. Generally, such portions of an antibody may be the antigen binding, variable region of an intact antibody or at least a portion of an antibody constant region such as the CH1, CH2, or CH3 regions. Examples of antibody derived binding proteins include Fab, Fab′, F(ab′)₂ and Fv fragments, diabodies, single chain antibody molecules and multispecific antibodies formed from at least two intact antibodies. Other examples include mimetibodies having the generic formula:
(V1(n)-Pep(n)-Flex(n)-V2(n)-pHinge(n)-CH2(n)-CH3(n))(m),
where V1 is at least one portion of an N-terminus of an immunoglobulin variable region, Pep is at least one bioactive peptide that binds to a second molecule, Flex is polypeptide that provides structural flexibility by allowing the mimetibody to have alternative orientations and binding properties, V2 is at least one portion of a C-terminus of an immunoglobulin variable region, phinge is at least a portion of an immunoglobulin variable hinge region, CH2 is at least a portion of an immunoglobulin CH2 constant region and CH3 is at least a portion of an immunoglobulin CH3 constant region, where n and m can be an integer between 1 and 10. A mimetibody mimics properties and functions of different types of immunoglobulin molecules such as IgG1, IgG2, IgG3, IgG4, IgA, IgM, IgD and IgE.

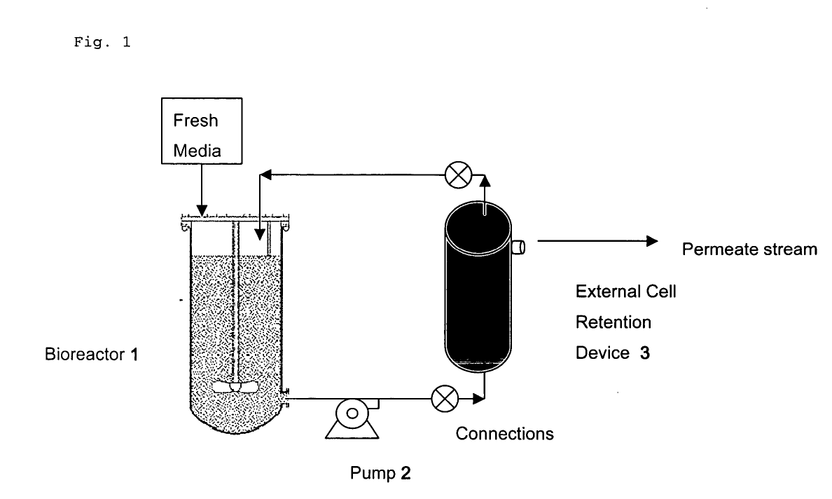

The term “bioprocessing system” as used herein means an essentially closed system for the production of a molecule of biological origin such as a polypeptide from a eukaryotic cell such as a mammalian or insect cell. A representative bioprocessing system configuration that may be used with the method of the invention is presented in FIG. 1 which shows the relationship between the bioreactor vessel 1, the recirculation pump 2 and an external cell retention device (CRD) such as an external spin filter (ESF) 3. The bioreactor is typically a 50 L to 2000 L volume vessel enclosing the reaction space, equipped with means for mixing and suspending the cell culture and capable of being completely sterilized in place.

Typically, the vessel will be a rigid stainless steel cylinder however, the vessel may, e.g., comprise a flexible polymeric container such as a cell bag. The bioreactor has feed lines for fresh medium and a removal line for drawing off a portion of the cell suspension. The removal line passes through a pump and continues through a connection, which may be sterilized in place, to the ESF. The ESF 3 also has connectors for connecting a line for harvested, essentially cell-free medium and a second line leading from the inner outlet at the point of cell concentration and back to the bioreactor. Valves are present at various points in the system to control flow and permit the sterilization of various components of the system.

The term “operating cell density” as used herein means that cell density at which a bioprocessing system will be operated to obtain the production of a molecule of biological origin. Such cell densities are those at which the nutrients such as amino acids, oxygen or other metabolites supplied to the bioprocessing system are sufficient to maintain cellular viability. Alternatively, such cell densities are those at which waste products can be removed from the bioprocessing system at a rate sufficient to maintain cellular viability. Such cell densities can be readily determined by one of ordinary skill in the art. In a typical bioprocessing system cell densities may be between about 0.5×10⁶ cells/ml and about 25×10⁶ cells/ml.

The term “permeate stream” as used herein means that portion of the media and suspended cells that exits the external CRD by passing through the retention barrier.

The term “retentate stream” as used herein means that portion of the media and suspended cells that exits the external CRD without passing through the retention barrier. Typically, the majority of cells is present in the retentate stream.

The present invention provides methods for maintaining a low shear environment thereby maintaining operating cell density in a bioprocessing system by minimizing fluid shearing stresses. Eukaryotic cells expressing a polypeptide such as an antibody or an antibody-derived binding protein or another protein of interest can be grown in the bioprocessing system. The methods of the invention are useful for extending operation time for the bioprocessing system thereby maximizing production time and the amount of product that can be recovered from the system. Further, the entire bioprocessing system can be sterilized in place thereby minimizing down time between bioprocessing runs.

In particular, the present invention provides methods for maintaining a low shear environment in a eukaryotic cell bioprocessing system by culturing a cell suspension in a vessel, removing a portion of the cell suspension from the vessel by the action of a peristaltic pump, delivering the portion of the suspension to a CRD that separates the suspension into a permeate stream and a retentate stream wherein the shear rate in the CRD is less than 3000 sec⁻¹, and returning the retentate stream to the vessel.

Continuous perfusion systems require agitation or movement in the bioreactor vessel to provide suspension of the cells, supply fresh nutrients and allow access to the fraction containing product. To obtain cell suspension, bioreactor vessels typically use one or more movable mechanical agitation devices that are a potential source of shear stress.

Examples of means for generating a cell suspension include impellers, such as propellers, or other mechanical means, bladders, fluid or gas flow-based means, ultrasonic standing wave generators, rocking platforms or combinations thereof which produce a cell suspension. In the methods of the invention, a propeller is an exemplary means for suspending the cells in the media and generating a shear rate of less than 20 s⁻¹. A propeller moves with a rotation speed (rpm) and has a diameter (D). A simplified calculation of the maximal shear force (Vt) which will occur tangentially to and at the tip of the propeller blade is the product of the blade radius and rotation rate such that:
Vt=radius×rotation rate=D/2×2Π×rpm.

Exemplary maximum shear rates produced by impeller agitators/bioreactor configurations useful in the methods of the invention are shown in Table 1.

TABLE 1
Shear rate of various large-scale perfusion bioreactors
based on the impeller tip speed

| | | | | | | Max. |
|---|---|---|---|---|---|---|
| | Bioreactor | Impeller | | | | Shear |
| Bioreactor | Diameter | Diameter | Gap | | Vt | Rate |
| Volume | (cm) | (cm) | (cm) | rpm | (cm/sec) | (/sec) |
| 100 L | 46 | 18 | 14 | 50 | 47 | 3 |
| 250 L | 26 | 10 | 8 | 60 | 31 | 4 |
| 500 L | 92 | 30 | 31 | 100 | 157 | 5 |
| 1000 L | 112 | 56 | 28 | 50 | 146 | 5 |

One of skill in the art could readily recognize additional vessels and means for generating a eukaryotic cell suspension within the vessel that are compatible with the method of the invention.

In the present invention, it has been determined that the type of pump used to move the cell suspension from the bioreactor to the CRD has a large affect on shear rate. In the method of the invention, shear rate is minimized by removing a portion of the eukaryotic cell suspension from the bioreactor vessel by the action of a peristaltic pump. Examples of such pumps include a Watson-Marlow (Falmouth, England) 600 series pump peristaltic pump, a Masterflex L/S series (Cole-Parmer, Barrington, Ill.) or other peristaltic pumps models.

Many different types of pumps are known in the art and include reciprocating pumps, rotary pumps, lobe pumps, centrifugal pumps, diaphragm pumps and peristaltic pumps. Lobe pumps have typically been employed in continuous perfusion bioprocessing systems. The lobe pump employs a lobed element or rotor for pushing liquid. There are generally only two or three lobes on each rotor. The two lobed elements are rotated, one directly driven by the source of power, and the other through timing gears. As the elements rotate, liquid is trapped between two lobes of each rotor and the walls of the pump chamber and carried around from the suction side to the discharge side of the pump. As liquid leaves the suction chamber, the pressure in the suction chamber is lowered, and additional liquid is forced into the chamber from the reservoir. The lobes are constructed so there is a continuous seal at the points where they meet at the center of the pump. The lobes of the pump are sometimes fitted with small vanes at the outer edge to improve the seal of the pump. The vanes are mechanically held in their slots, but with some freedom of movement. Centrifugal force keeps the vanes snug against the chamber and the other rotating members.

The structure of a lobe pump provides a gap between the walls of the pump chamber and the lobe element at certain points during its rotation resulting in shear stress on cell-containing culture media passing through the pump. For example, with a pump chamber diameter of 6.46 cm and a lobe diameter of 6.35 cm, the gap through which the cells must pass fluctuates between 0 and 0.11 cm as the lobe rotates. Shear rates in excess of 3000 sec⁻¹ typically damage cells, especially in the absence of animal-product derived cell protectants such as primatone and/or serum.

Peristaltic pumps work on the principle of sequential narrowing of the diameter of a shaft or portion of tubing in order to move liquid along the length of the tubing. The fluid is totally contained within a tube or hose and does not come into contact with the pump. These pumps have no seals, glands or valves and thus are ideal for hygienic or sterile operation. Peristaltic pumps are equally successful in pumping slurries and sludges without clogging or blockage due to their straight flow path. Being true positive displacement pumps, there is no slip or back flow.

The peristaltic pump may engage tubing made of a composite material. One example of such tubing is Sta-Pure® pump tubing (Mitos Technologies, Inc., Phoenixville, Pa.) which is made from a composite material comprising a silicon polymer and polytetrafluoroethylene (PTFE; also known as Teflon®). Other examples of composite tubing suitable for use with the method of the invention include fiber reinforced polymeric tubing. These configurations provide for sterilization in place of the complete bioprocessing system. Those of skill in the art will recognize other peristaltic pumps and tubing compatible with the method of the invention.

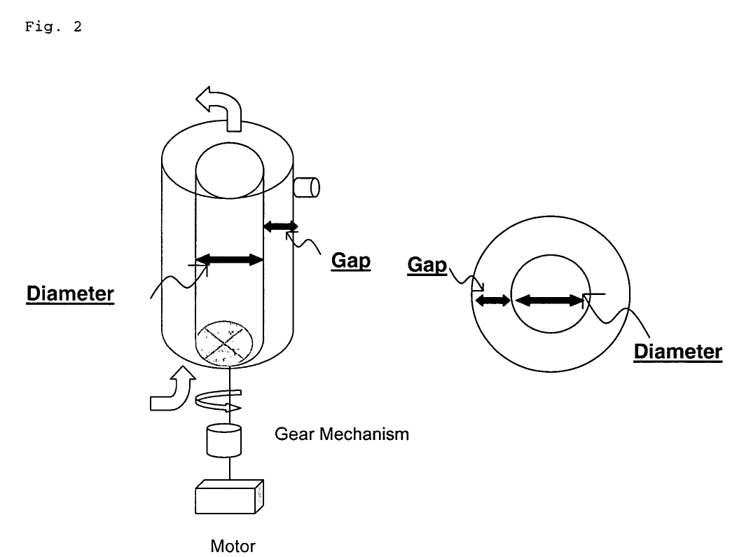

Shear stress can also be generated in the CRD unit of the bioprocessing system. For example, in an ESF, the device comprises a tank housing of a given inner diameter (d) and a spin filter basket with a second diameter holding a screen (See FIG. 2). There is a gap distance between the tank inner wall and the spin filter basket/screen and the ratio between the diameters of the tank inner wall and the basket/screen is defined as kappa (k). Calculation of shear rate for the ESF component is based on the rotational speed of the basket (Vt) and the distance (L) along the gap and can be calculated based on Atsumi's correlation. See Choi et al., J. Membr. Sci. 157, 177-187 (1999).

Typically the ESF diameter is designed in such a way as to minimize the gap between the ESF tank and the spin filter to preserve turbulence. Turbulence has been considered essential in preventing filter clogging. However, one can reduce the shear rate of the ESF system by reducing shear rate contribution through reduction in gap size. The applicants have unexpectedly found that by reducing the speed of rotation of the basket while keeping gap size minimized, shear was reduced with no increase in filter clogging.

Another approach to reduce shear from the gap is to reduce ESF diameter. Various reduced diameters can be fabricated to serve such purposes. Table 2 shows the significant shear stress contributions from ESF gaps and ESF basket speed for various bioreactor configurations.

TABLE 2
ESF shear stress contributions for various bioreactors.

| | | | k (ratio | | | |
|---|---|---|---|---|---|---|
| | ESF | Spin | of ESF | | | Shear |
| Bioreactor | Tank D | Filter D | tank/spin | | Vt | Rate |
| volume | (cm) | (cm) | filter) | rpm | (cm/sec) | (/sec) |
| 30 L w/ESF | 6.7 | 5.8 | 0.85 | 650 | 196 | 7632 |
| 100 L w/ESF | 21.8 | 20.4 | 0.94 | 72 | 77 | 713 |
| 250 L w/ESF | 25.4 | 20.4 | 0.80 | 73 | 78 | 733 |
| 250 L w/ | 25.4 | 13.0 | 0.51 | 73 | 50 | 298 |
| reduced | | | | | | |
| diameter | | | | | | |
| ESF | | | | | | |

In the method of the invention the portion of the eukaryotic cell suspension removed from the bioreactor is delivered to an external spin filter so as to separate the suspension into a retentate stream and a permeate stream. The retentate stream is then returned to the vessel of the bioprocessing system for further culturing.

In the methods of the invention, shear rates generated by the CRD are below 3000 s⁻¹, below 2000 sec-1 or below 1500 sec-1. An exemplary ESF shear rate range during a bioprocessing system production run is between about 1235 s⁻¹ and about 700 s⁻¹. To keep the ESF shear rates in this range, the ESF rotation speeds are typically from about 25 to about 300 rpm, the diameter is about 5 to about 30 cm and the gap is about 0.5 to about 5 cm.

The eukaryotic cells cultured in the method of the invention may be any cell line capable of growth under continuous perfusion culture conditions. These cells include myeloma derived cell lines such as, e.g., NSO cells, Sp2/0 cells, Ag653 cells (American Type Culture Collection Accession No. ATCC CRL 1580) or other myeloma derived cell lines and Chinese Hamster Ovary (CHO) cell lines known to those skilled in the art.

The method of the present invention can also be used to maintain a low shear environment in a bioprocessing system for periods of time ranging from 20 days to more than 40 days. An exemplary operating time is at least about 30 days. Operating cell densities that may be maintained are those from at least about 0.5×10⁶ cells/ml. In a typical bioprocessing system operating cell densities may be between about 0.5×10⁶ cells/ml and about 25×10⁶ cells/ml. Exemplary densities can be between about 2.5×10⁶ cells/ml and about 22×10⁶ cells/ml. In the method of the invention, cell viability is typically between about 40% and about 100%. Other bioprocessing system operating cell densities and acceptable cell viability levels will be recognized by those skilled in the art and can be determined by techniques well known to those of skill in the art.

The present invention will now be described with reference to the following specific, non-limiting examples.

### EXAMPLE 1

### Use of Large-Scale Peristaltic Pump to Reduce Shear in a Bioprocessing System

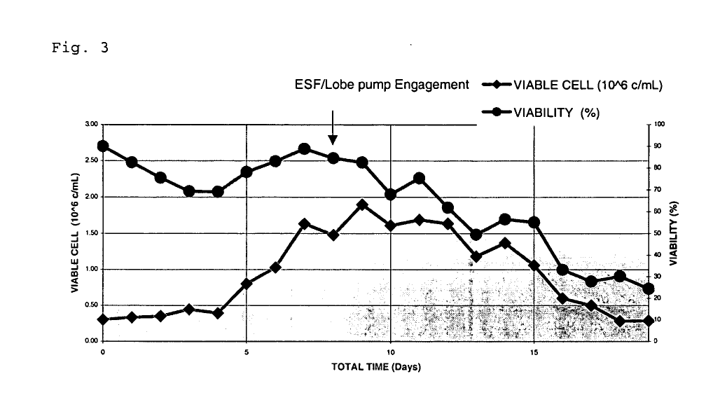

A shear sensitive NSO cell line expressing an anti-CD3 antibody (described in US Pat. No. 6,491,916) was grown in the presence of serum in a continuous perfusion bioreactor using a lobe pump recirculator. These cells were damaged by the bioprocessing system when the lobe pump was used for recirculation and the delivery of cell suspension to the ESF. The result was an unacceptably low viability of 20% after 12 days of bioprocessing system operation (FIG. 3).

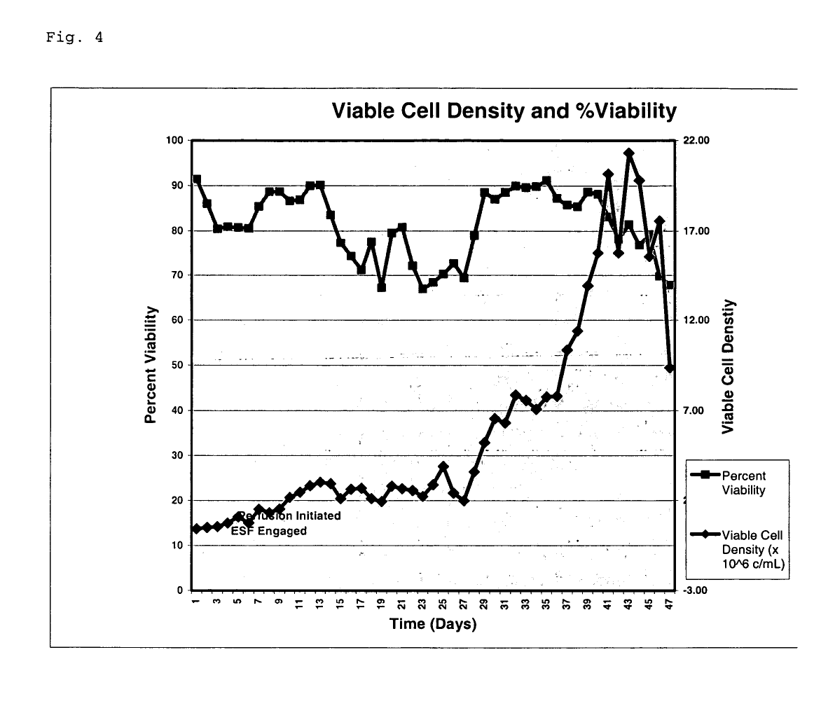

Consequently, the propeller used for generating a cell suspension in the perfusion bioreactor was operated such that the shear rate of between 10 s⁻¹ and 20 s⁻¹ was maintained. Additionally, the lobe pump was replaced with a Watson-Marlow (Falmouth, England) 600 series peristaltic pump to reduce shear. After replacing lobe pump with the peristaltic pump, the results in FIG. 4 show that cell growth and viability could be sustained in the bioprocessing system for at least 40 days without ESF filter material change out.

### EXAMPLE 2

### Reduction of ESF Rotation Speed

Typical operating conditions in an ESF used for large-scale production contributes to the shear rate. The results in Table 3 show that in small-scale optimization experiments, a tip speed of 78 cm s⁻¹ produces an acceptable shear rate of 1229 s⁻¹. Keeping tip speed constant at 78 cm s⁻¹ in a 100 L scale up bioreactor configuration, the rotational speed of the ESF is reduced approximately 25% and the corresponding shear rate is 735 sec⁻¹.

TABLE 3
Reduction of ESF Rotational Speed to Reduce Shear Stress

| | | Tip | Shear | |
|---|---|---|---|---|
| ESF Condition based on | | Speed | Rate | 100 L Bx ESF |
| Reduced Shear | Speed | (cm s⁻¹) | (s⁻¹) | Spin Speed % |
| Small Scale Bioreactor | 260 | 78.3 | 1229 | NA |
| 100 L Scale-Up based on | 72 | 78.0 | 735 | 25% |
| Tip Speed | | | | |
| 100 L Scale-Up based on | 103 | 110.0 | 1220 | 37% |
| Shear Rate | | | | |

The present invention now being fully described, it will be apparent to one of ordinary skill in the art that many changes and modifications can be made thereto without departing from the spirit or scope of the appended claims.

1. A method for maintaining a low shear environment in a eukaryotic cell bioprocessing system comprising the steps of:
(a) culturing a cell suspension in a vessel;
(b) removing a portion of the suspension from the vessel by the action of a peristaltic pump;
(c) delivering the portion of the suspension to an external cell retention device (CRD) that separates the suspension into a permeate stream and a retentate stream wherein the shear rate in the external CRD is less than 3000 sec⁻¹; and
(d) returning the retentate stream to the vessel.

2. The method of claim 1 wherein the CRD is a spin filter.

3. The method of claim 1 wherein the cell suspension is cultured in the absence of animal-derived cell protectants.

4. The method of claim 1 wherein the vessel comprises a means for generating a cell suspension that produces a shear rate below 20 sec⁻¹.

5. The method of claim 1 wherein the CRD shear rate is less than 2000 sec⁻¹.

6. The method of claim 1 wherein the CRD shear rate is less than 1500 sec⁻¹.

7. The method of claim 1 wherein the operating cell density is maintained at up to about 25×10⁶ cells/ml.

8. The method of claim 6 wherein the operating cell density is maintained for at least about 30 days.

9. The method of claim 1 wherein the eukaryotic cell suspension comprises cells secreting a polypeptide.

10. The method of claim 9 wherein the polypeptide is an antibody or antibody-derived binding protein.

11. The method of claim 9 wherein the cell suspension is myeloma cells.

12. The method of claim 11 where in the myeloma cells are NSO cells.

13. The method of claim 1 wherein the bioprocessing system is sterilizable in place.

14. A method for maintaining an operating cell density of up to about 25×10⁶ cells/ml in a bioprocessing system for at least 20 days, comprising the steps of:
(a) culturing a myeloma cell suspension capable of secreting a polypeptide in a vessel with a volume of at least 50 L;
(b) removing a portion of the suspension from the vessel by the action of a peristaltic pump;
(c) delivering the suspension to an external spin filter so as to separate the suspension therein into a permeate stream and a retentate stream where the external spin filter generates a shear rate below 1500 s⁻¹; and
(d) returning the retentate stream to the vessel.

15. The method of claim 14 wherein the polypeptide is an antibody or an antibody-derived binding protein.

16. The method of claim 14 wherein the myeloma cells are NSO cells.
